(19) United States
(12) Patent Application Publication
(10) Pub. No.: US 2021/0009844 A1
(43) Pub. Date: Jan. 14, 2021

(54) COALESCING AGENTS FOR WATERBORNE COATINGS

(71) Applicant: Arkema Inc., King of Prussia, PA (US)

(72) Inventors: Wenjun Wu, Cary, NC (US); Jeffrey A. Schneider, Holly Springs, NC (US); Zuzanna Donnelly, Ardmore, PA (US); Alex R. Pederson, Owatonna, MN (US); Jeffrey P. ARENDT, Cary, NC (US); An Du, Blue Bell, PA (US)

(21) Appl. No.: 16/969,574

(22) PCT Filed: Feb. 18, 2019
(86) PCT No.: PCT/US2019/018398

§ 371 Date: Aug. 13, 2020

Related U.S. Application Data
(60) Provisional application No. 62/631,966, filed on Feb. 19, 2018.

(51) Int. Cl. C09D 133/08; C07D 303/42

(52) U.S. Cl. CPC C09D 133/08; C08K 5/1515; C07D 303/42

(57) ABSTRACT

Compounds comprising one or more functionalized fatty acid esters, which may be derived from bio-based oils, are used as a low-VOC coalescent agent (i.e., a coalescent agent having a low content of volatile organic compounds) in waterborne coating compositions. The functional group can be epoxide, vicinal diol, hydroxy phosphotriester, hydroxy ester, hydroxyl alkyl ester, hydroxyl benzyl ester, hydroxy ether, hydroxy amino, hydroxy sulfide, hydroxy nitrile, hydroxy amine, terminal alcohol, thiiran, ketone, or cyclic carbonate. The present disclosure also relates to waterborne coating compositions comprising these functionalized fatty acid esters.

### FIELD OF THE INVENTION

Compounds comprising one or more functionalized fatty acid esters, which may be derived or prepared from bio-based oils such as vegetable and animal oils, are used as a low-VOC coalescent agent (i.e., a coalescent agent having a low content of volatile organic compounds) in waterborne coating compositions. The functional group(s) in the functionalized fatty acid ester(s) can be epoxide, vicinal diol, hydroxy phosphotriester, hydroxy ester, hydroxyl alkyl ester, hydroxyl benzyl ester, hydroxy ether, hydroxy amino, hydroxy sulfide, hydroxy nitrile, hydroxy amine, terminal alcohol, thiiran, ketone and cyclic carbonate. The present disclosure also relates to waterborne coating compositions comprising these functionalized fatty acid esters. The waterborne coating compositions comprise a polymeric resin emulsion of polymers such as vinyl acetate homopolymers, vinyl acetate copolymers, acrylic homopolymers, acrylic copolymers, vinyl acetate ethylene copolymers, fluoropolymers and acrylic modified fluoropolymers, or styrene acrylic copolymers.

### BACKGROUND OF THE INVENTION

The coalescing agent is an important component of water-based latex or emulsion coating compositions. As the coating dries after being applied to a substrate, the purpose of the coalescent agent in these compositions is to aid the discrete particles of polymer that are present in the latex to form into a continuous film. The coalescent agent also can contribute to a good performance balance of various coating properties.

Traditionally, these coalescing agents have been comprised of volatile compounds. A purpose of coalescing aids generally is to temporarily plasticize, i.e., lower the glass transition temperature (Tg), of emulsion polymers. The lower Tg allows the polymers to coalesce, i.e., form into a continuous film, at lower temperatures, which is desirable. However, lowering the Tg also tends to lower the hardness of the coating, which can degrade durability. The volatile coalescing aids leave the coating film by evaporation after film formation and thus the polymer regains its original Tg and hardness.

However, the volatile coalescing agent may be a significant contributor to the volatile organic compound “VOC” content of a waterborne coating. Environmental regulations and consumer awareness continue to push demand for environmentally friendly waterborne coatings having low VOC content and that preferably utilize bio-sourced and/or renewable raw materials, while still maintaining the performance of conventional coatings made with high-VOC coalescent agents. Because of demand for low-VOC coalescent agents, many low-VOC or zero-VOC coalescents have been developed in recent years for use in waterborne coatings, see for example: U.S. Pat. Nos. 8,383,710; 8,586,777; 9,034,964; 9,169,372; 9,193,843; 9,758,637; US 2009/0151601; and US 2015/0025167.

A significant drawback of low-VOC coalescents is that due to their low volatility they tend to stay in the coating films for a prolonged period of time and can therefore compromise some of the coating properties, most often blocking resistance.

Further, an ideal coalescent aid should have low water solubility but should still disperse or dissolve in latexes/emulsions useful in paint and coating formulations. The effectiveness of the coalescent aid in waterborne coatings generally depends on its solubility in water and compatibility with the polymer. In addition, a preferred coalescent agent resists yellowing over time.

A modification of natural oil derivatives has been tried, for example, as described in “Preparation of Glycol Esters of Soybean Oil Fatty Acids and Their Potential as Coalescent Aids in Paint Formulations” (JAOCS, vol. 77, no. 7, pp. 691-697 (2000)) which discloses the preparation of soy oil glycol monoesters through transesterification of soybean oil with glycols. The resulting composition of soy oil glycol esters can be used in water-based paint formulations as a coalescent aid to reduce minimum film formation temperature.

Epoxidized natural oils and/or epoxidized fatty acid alkyl esters have been used as plasticizers at high concentrations to produce flexible PVC. When used in PVC, these materials are generally added via a dry compounding process and therefore compatibility with water is not an issue. Other efforts to develop environmentally friendly coatings or coating compositions have been focused on various polymers and various coalescent agents, but notably these compositions utilize polymers that are not present as a waterborne emulsion, or do not use natural oils or derivatives of natural oils as a low-VOC coalescent in a waterborne coating formulations.

U.S. Pat. No. 9,034,965, which is incorporated herein by reference for all purposes, discloses an epoxidized composition and a process for producing the same. The epoxidized blend is useful for plasticizing a polymer composition comprising homopolymers or copolymers of polyvinyl chloride (PVC).

U.S. Pat. No. 9,238,728, which is incorporated herein by reference for all purposes, claims a composition consisting of a blend of a) at least one biodegradable thermoplastic material that includes poly(lactic acid); and b) at least one plasticizer that includes an epoxidized fatty acid alkyl ester.

U.S. Patent Application Publication No. 2015/0368431 relates to a plasticizer composition comprising epoxidized fatty acid alkyl ester (eFAAE); and an epoxidized natural oil, wherein at least a portion of the eFAAE is derived from a natural-oil soap stock and at least a portion of the natural-oil soap stock is derived from soybean oil. The plasticizer composition is useful as plasticizer for polymeric compositions comprising polyvinyl chloride (PVC).

WO Patent Publication 2017/123578 A1, the contents of which are incorporated by reference herein for all purposes, is related to phthalate-free, epoxidized plasticizer compositions for use in polyvinylchloride polymers. The plasticizer compositions comprise a blend of one or more fatty acid esters and one or more bio-based oils, and methods of making the same.

U.S. Pat. No. 6,797,753 claims a plasticized polyvinyl chloride (PVC) composition, comprising a) about 100 parts by weight of at least one vinyl chloride resin; b) about 10-230 parts by weight of a primary plasticizer comprising a fatty acid derived from a vegetable oil having at least 80% by weight of unsaturated fatty acids, wherein the unsaturated fatty acids are fully esterified with a mono alcohol or polyol, and the esterified unsaturated fatty acids are fully epoxidized.

U.S. Pat. No. 5,846,601 claims a method for soil stabilization comprising applying a biodegradable aqueous polymer dispersion to a surface layer of soil, wherein the biodegradable aqueous polymer dispersion comprising a polyvinyl acetate polymer and a biodegradable plasticizer member selected from the group consisting of triesters of glycerol with lower aliphatic monocarboxylic acids, citric acid triesters with lower aliphatic monohydric alcohols, epoxidized triglycerides of at least partly olefinically unsaturated fatty acids, and mixtures of two or more of such members.

U.S. Patent Application Publication No. US 2012/0258249 A1 relates to glycol ether-esters used as coalescent agents for aqueous polymeric dispersions.

U.S. Patent Application Publication No. US 2009/0151601 A1 relates to the use of fatty acid esters as low-VOC coalescent aids for water based coatings that also improve the efflorescence resistance of the coating. The disclosed fatty acid esters have the formula RCOOX wherein R is a hydrocarbyl having one or more aliphatic carbon-carbon double bonds, and X is selected from the group consisting of a saturated hydrocarbyl, a hydrocarbyl having one or more aliphatic carbon-carbon double bonds and a substituted hydrocarbyl. Epoxidized fatty acid esters are not disclosed.

International Application Publication No. WO 00/56823 discloses generally a film-forming composition comprising a particulate polymer or emulsified liquid pre-polymer, water, and a coalescent aid comprising an ester having the formula RCOOX wherein R and X are independently hydrocarbyl or substituted hydrocarbyl, and at least one of R and X contain at least two unsaturated carbon-carbon bonds. These soy oil glycol monoesters are prepared by transesterification of soybean oil with glycols. The resulting composition of soy oil glycol esters can be used in water-based paint formulations as a coalescent aid to reduce minimum film formation temperature.

While those skilled in the art would have expected that long chain hydrocarbon compounds would be too hydrophobic or too bulky to be useful as coalescent agents in water-borne emulsions, surprisingly the functionalized fatty acid ester coalescent agents of the present invention were found to have good compatibility and coalescing efficiency with the polymeric resins typically employed in waterborne coating formulations, including vinyl acetate homopolymers and copolymers, all acrylic polymers, styrene acrylic emulsion polymers, and acrylic fluoropolymer blends. Compared to commercially availabe low-VOC coalescent products, the coating compositions described herein, comprising bio-based, low-VOC coalescents that can be derived from natural oils, have a more preferred environmental profile and exhibit equivalent or better coating performance than comparative commercialized low VOC coalescents.

Emulsion polymers and monomers useful to prepare polymeric emulsions or dispersions in which these low-VOC coalescent agents can be used are known in the art (in texts on the subject such as “Emulsion Polymerization: Theory and Practice” by D. C. Blackley published by Wiley in 1975, “Emulsion Polymerization” by F. A. Bovey et al. published by Interscience Publishers in 1965, and “Emulsion Polymerization and Emulsion Polymers” by P. A. Lovell et al. published by Wiley Science in 1997).

### SUMMARY OF THE INVENTION

The invention relates to low volatility coalescent agents for waterborne coating compounds. These novel coalescent agents are functionalized fatty acid esters that can be prepared from natural oils. Blends of these materials are also part of the scope of the present disclosure. The functional groups can be selected from epoxides, vicinal diols, hydroxy phosphotriesters, hydroxy esters, hydroxyl alkyl esters, hydroxyl benzyl esters, hydroxy ethers, hydroxy aminos, hydroxy sulfides, hydroxy nitriles, hydroxy amines, terminal alcohols, thiiran, ketones, or cyclic carbonates. Importantly, these additives do not degrade the physical properties of the final coating.

These low volatility coalescent agents can be used in waterborne coating compositions comprising a wide variety of polymers that are normally used in waterborne emulsion or latex coating compositions.

### BRIEF DESCRIPTION OF THE DRAWINGS

FIG. 1 is a schematic showing possible reaction products of the ring-opening reaction of epoxides that may be used in accordance with certain embodiments of the invention.

### DETAILED DESCRIPTION OF THE INVENTION

The invention relates to functionalized fatty acid esters derived from natural oils that are used as low-VOC coalescent agents in waterborne coating compositions. The functional groups on these fatty acid esters may be epoxide, vicinal diol, hydroxy phosphotriester, hydroxy ester, hydroxyl alkyl ester, hydroxyl benzyl ester, hydroxy ether, hydroxy amino, hydroxy sulfide, hydroxy nitrile, hydroxy amine, terminal alcohol, thiiran, ketone, and/or cyclic carbonate, or a combination thereof. A preferred embodiment is a coalescent agent that is a functionalized fatty acid ester prepared from natural oils where the functional group is selected from epoxides. The epoxide-functionalized fatty acid esters can be conveniently prepared by epoxidizing fatty acid esters that comprise some unsaturation, according to any method known in the art. The other functional groups may then be conveniently prepared by the ring opening reactions of the epoxide groups present in the epoxide-functionalized fatty acid esters.

It is worth emphasizing that while certain of these molecules may be conveniently described as the reaction product of ring opening reactions of the corresponding epoxidized compound, they may be prepared by any other method as known in the art. While the epoxidized materials are conveniently made by epoxidizing molecules containing at least some degree of unsaturation, they too may be made by any other method as known in the art. These materials can be blended together by any means known in the art in all proportions to be used as coalescent agents in waterborne coating compositions. Further, blends can be made by blending the un-epoxidized compounds and then subjecting the resultant mixture to suitable conditions to effect epoxidation.

Unless otherwise indicated, all percentages herein are weight percentages,

“Polymer” as used herein, is meant to include organic molecules with a weight average molecular weight higher than 20,000 g/mol, preferably higher than 50,000 g/mol, as measured by gel permeation chromatography.

Coalescent Agents

Suitable coalescent agents include functionalized fatty acid esters. The term “fatty acid ester” refers to compounds that result from the reaction (esterification) of an alcohol with a fatty acid. They can be fatty acid monoesters (e.g., fatty acid monoglycerides, fatty acid esters of aliphatic mono-alcohols such as methanol or ethanol, aromatic mono-alcohols such as benzyl alcohol), fatty acid diesters (e.g., fatty acid diglycerides, fatty acid esters of diols such as glycols in which both hydroxyl groups are esterified with fatty acid), fatty acid triesters (e.g., fatty acid triglycerides, fatty acid esters of triols other than glycerin in which all three hydroxyl groups are esterified with fatty acid) and fatty acid esters of polyols containing more than three hydroxyl groups per molecule. Preferred such compounds comprise at least 14 carbon atoms in total. The fatty acid moieties present in such compounds may be, for example, C8 to C26 fatty acid moieties, containing (prior to epoxidation) at least some amount of unsaturation, such as may be provided by an unsaturated fatty acid moiety, including both mono- and polyunsaturated fatty acid moieties such linoleic acid moieties, linolenic acid moieties, oleic acid moieties, elaidic acid moieties, erucic acid moieties, palmitoleic acid moieties, and the like. Where the compound contains more than one fatty acid moiety per molecule (as in the case of fatty acid diglycerides and fatty acid triglycerides, for example), all of the fatty acid moieties may be unsaturated fatty acid moieties or the compound may contain both unsaturated fatty acid moieties and saturated fatty acid moieties (e.g., stearic acid moieties, palmitic acid moieties, lauric acid moieties and the like) provided the compound contains at least one unsaturated fatty acid moiety.

A coalescent agent composition in accordance with the present invention may comprise, in addition to at least one functionalized fatty acid ester, one or more non-functionalized fatty acid esters (i.e., fatty acid esters that do not contain any of the aforementioned functional groups).

Typically, but not necessarily, fatty acid esters are natural oils obtained from plant or animal sources (as used herein, the term “oils” refers to fatty acid triglycerides, regardless of whether they are liquid or solid at 25° C.). They may also be interesterified oils prepared from mixtures of oils, including natural oils. Non-limiting examples of suitable natural oils from which fatty acid esters can be prepared are: algae oil, avocado oil, canola oil, coconut oil, castor oil, corn oil, cottonseed oil, flax oil, fish oil, grapeseed oil, hemp oil, jatropha oil, jojoba oil, mustard oil, dehydrated castor oil, palm oil, palm stearin, rapeseed oil, safflower oil, soybean oil, sunflower oil, tall oil, olive oil, tallow, lard, chicken fat, linseed oil, tung oil, linoleic oil, peanut oil, coconut oil and mixtures thereof. It is to be understood that while these materials are most conveniently derived from such examples of natural sources as listed above, that such structures synthesized by any other means are also envisioned as part of this disclosure.

Preferred coalescent compositions comprise epoxidized fatty acid alkyl esters of fatty acids obtained from vegetable or animal oils, with alkyl being C1 to C22, or C1 to C8, or C2 to C8. Such compounds may correspond to the general formula RC(═O)OR′, wherein R is a long chain aliphatic group containing one or more epoxide functional groups and R′ is a C1 to C8 alkyl group, and preferably a C2 to C8 alkyl group. The preferred minimum chain length for the fatty acid portion(s) of the molecule (RC(═O)O—) is 14 carbon atoms.

Also suitable as coalescent agents are epoxidized fatty acid benzyl esters of fatty acids obtained from vegetable or animal oils (where R′ in the aforementioned formula is —CH₂—Ar, with Ar being a benzene ring). A non-limiting example of such a compound is the reaction (i.e. esterification) product of a vegetable oil (or fatty acid obtained from a vegetable oil) with benzyl alcohol, for instance.

Also suitable as coalescent agents and included in the scope of this disclosure are the functionalized products that can be prepared by ring-opening reactions of any of the epoxidized compounds described herein (e.g., the above-mentioned epoxidized fatty acid esters).

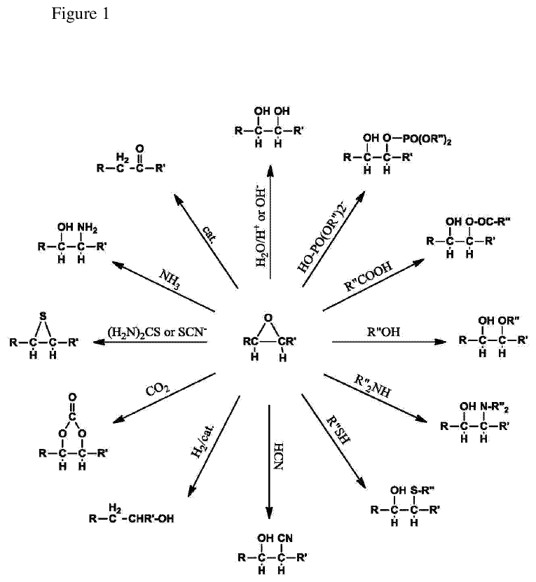

FIG. 1 shows the ring-opening reactions of the epoxide groups on these epoxidized molecules that produce the functionalized compounds that can be used as coalescent agents in waterborne coating compositions according to the present disclosure. The resulting molecules comprise at least one group for each epoxide ring that was present on the corresponding epoxidized molecule and that has been ring-opened. Generally, most have a hydroxyl group adjacent to (i.e., separated by two carbon atoms) a second group such as: a second hydroxyl group, phosphotriester, ester, ether, amino, sulfide, nitrile, or amine, depending on the reactants. These can be referred to as vicinal diols, hydroxy phosphotriesters, hydroxy ester, hydroxy ether, hydroxy amino, hydroxy sulfide, hydroxy nitrile, or hydroxy amine, respectfully. Other groups that do not have an adjacent hydroxyl group are: terminal alcohol, ketone, thiiran or cyclic carbonate. It should be understood that while these functionalized molecules may be conveniently described as the reaction product of ring opening reactions of the corresponding epoxidized compound, they may be prepared by any other method as known in the art. It is also to be understood that the epoxidized molecules, while conveniently made by epoxidizing a molecule containing at least some unsaturation, also can be made by any other method as known in the art.

It is to be understood that complete epoxidation of any of these compounds is not necessary in the practice of the invention, nor is complete ring-opening of all of the epoxy groups in such compound(s) when forming the functionalized groups. Suitable oxirane values for a coalescent agent composition in accordance with the present invention can range from 0 (for fully ring-opened compounds) to 10 weight percent of oxirane oxygen as measured by the method described in Analytical Chem., No. 36, 1964, pp. 667-668. If the functional groups comprise epoxide groups, suitable oxirane values are between 4 and 10 weight percent of oxirane oxygen. While residual unsaturation is not a detriment to the utility of these materials, the iodine number (also referred to as “iodine value”) of the preferred compounds should be no more than 10 g I₂/100 g or no more than 4 g I₂/100 g or no more than 2 g I₂/100 g as measured by AOCS Cd 1b-87. Residual acid from the epoxidation reaction (resulting from esterification and/or epoxidation reactions, for example) should preferably be no more than 10 mg KOH/g, and more preferably less than 5 mg KOH/g as measured by AOCS Te 2a-64.

Also suitable for use as coalescent agents are the products of partially epoxidized compounds resulting from the reaction of epoxidized fatty acid esters with straight chain or branched acids or alcohols. These are shown as the intermediates in the reactions disclosed in U.S. Pat. No. 9,586,918, the entire disclosure of which is incorporated by reference herein for all purposes.

A preferred coalescent composition comprises epoxidized fatty acid alkyl esters of fatty acids obtained from vegetable or animal oils, with alkyl being C1 to C22, or preferably C1 to C8, or more preferably C2 to C8, or a combination thereof, excluding coalescent compositions of epoxidized fatty acid alkyl esters obtained from soybean oil with alkyl being Cl (that is, methyl epoxy esters derived from soybean oil) as the functionalized fatty acid ester component. Functionalized, mono, di- and triglycerides (wherein glycerin is substituted with one, two or three fatty acid moieties) are also suitable.

It is to be understood that blends of these coalescent agents in all proportions are considered to be part of the invention. Also part of the invention are blends of ranges of these compounds. Non-limiting examples are, for instance, blends in all proportions of a range of molecular weights of and/or blends of various functionalized natural oil derivatives as described above.

These coalescent agents can be blended into waterborne latex or emulsion coating compositions at levels ranging from 1% to 20%, 2% to 15%, 3% to 10%, or 2% to 8% by weight of the dry polymer content in the composition.

Polymers in Latex Coating Compositions

Suitable waterborne coatings where the inventive coalescent agent can be utilized include architectural and industrial coatings, original equipment manufacturer coatings, special purpose coatings, lacquers, varnishes, enamels, caulks and sealants, inks, and other polymeric coatings where plasticizers and coalescents are traditionally used.

Emulsion polymers and monomers useful to prepare polymeric emulsions or dispersions are known in the art (in texts on the subject such as “Emulsion Polymerization: Theory and Practice” by D. C. Blackley published by Wiley in 1975, “Emulsion Polymerization” by F. A. Bovey et al. published by Interscience Publishers in 1965, and “Emulsion Polymerization and Emulsion Polymers” by P.A. Lovell et al. published by Wiley Science in 1997).

The coalescent compositions of the present invention are useful in waterborne coating compositions comprising a wide variety of polymers, which include but are not limited to: various vinyl polymers, such as polyvinyl chloride and copolymers thereof, poly(vinyl acetate) and copolymers thereof; vinyl acetate ethylene copolymers, various polyacrylates and copolymers thereof (e.g., polymers prepared from monomers such as methyl (meth)acrylate, ethyl (meth)acrylate, butyl (meth)acrylate, cyclohexyl (meth)acrylate, allyl methacrylate, 2-ethylhexyl acrylate; various acrylic acids such as methacrylic acid, acrylic acid, itaconic acid, etc), and various esters of versatic acid and copolymers; polystyrene and styrenated acrylic polymers (e.g., polymers of styrene and/or alpha-methyl styrene and copolymers of styrene and/or alpha-methyl styrene with alkyl (meth)acrylate and acid monomers). Acrylic polymers, as used herein, include but are not limited to homopolymers, copolymers, and terpolymers comprising alkyl (meth)acrylates.

Other methacrylate, acrylate, and other vinyl monomers, e.g. vinyl cyanide monomers and acrylonitrile, useful in the monomer mixture include, but are not limited to methyl acrylate, ethyl acrylate and ethyl methacrylate, butyl acrylate and butyl methacrylate, iso-octyl methacrylate and acrylate, lauryl acrylate and lauryl methacrylate, stearyl acrylate and stearyl methacrylate, isobornyl acrylate and methacrylate, methoxy ethyl acrylate and methacrylate, 2-ethoxy ethyl acrylate and methacrylate, and methacrylate monomers, styrene and its derivatives, acrylonitrile, and vinyl cyanides.

Also useful in the preparation of suitable emulsion polymers that can be used in the practice of this invention are functional co-monomers such as acid co-monomers, silane co-monomers, wet adhesion co-monomers, crosslinking and crosslinkable co-monomers, including the following non-limiting examples.

Acid co-monomers include but are not limited to (meth)acrylic acid, maleic acid, fumaric acid, itaconic acid, ethacrylic acid, crotonic acid, citraconic acid, cinnamic acid, phthalic acid, isophthalic acid, terephthalic acid, tetrahydrophthalic acid, hexahydrophthalic acid, tetrabromophthalic acid, trimellitic acid, pyromellitic acid, 1,4,5,6,7,7-hexachloro-5-norbornene-2,3-dicarboxylic acid, succinic acid, 2,6-naphthalenedicarboxylic acid, glutaric acid, sebacic acid, azelaic acid, 1,4-cyclohexanedicarboxylic acid, and 1,3-cyclohexanedicarbocylic acid.

A strong acid co-monomer selected from phosphorus-based or sulfur-based acid monomers or phosphate co-monomers may be used, including non-limiting examples such as: phosphoalkyl (meth)acrylates or acrylates; phospho alkyl (meth)acrylamides or acrylamides; phosphoalkyl crotonates, phosphoalkyl maleates, phosphoalkyl fumarates, phosphodialkyl (meth)acrylates, phosphodialkyl crotonates, vinyl phosphates or (meth)allyl phosphate; phosphate esters of polypropylene glycol mono(meth)acrylate or polyethylene glycol mono(meth)acrylate; polyoxyethylene allyl ether phosphate, or vinyl phosphonic acid.

Sulfate-based co-monomers include, without limitation, vinyl- and allyl-sulfonic or sulfuric acids; sulfoethyl (meth)acrylate, aryl-sulfonic or sulfuric acids; (meth)acrylamidoethane-sulfonic or sulfuric acids; methacrylamido-2-methyl propane-sulfonic or sulfuric acids; and the alkali metal salts of sulfonic and sulfuric acids.

Nitrogen-containing wet adhesion co-monomers include but are not limited to: ureido (meth)acrylates, (meth)acrylates with at least one of urea and thiourea in the side chains; acrylic allophanes, aminoethyl acrylate and methacrylate; dimethylaminoethyl acrylate and methacrylate; diethylaminoethyl acrylate and methacrylate, dimethylaminopropyl acrylate and methacrylate; 3-dimethylamino-2,2-dimethylpropyl acrylate and methacrylate; 2-N-morpholinoethyl acrylate and methacrylate; 2-N-piperidinoethyl acrylate and methacrylate; N-(3-dimethylaminopropyl)acrylamide and -methacrylamide; N-dimethylaminoethylacrylamide and -methacrylamide; N-diethylaminoethylacrylamide and -methacrylamide; N-(4-morpholinomethyl)acrylamide and -methacrylamide; vinylimidazole and also monoethylenically unsaturated derivatives of ethyleneurea, such as N-(2-(meth)acryloyloxyethyl)ethyleneurea, N-(β-acrylamidoethyl)ethyleneurea, N-2-(allylcarbamato)aminoethylimidazolidinone, N-vinylethyleneurea, N-(3-allyloxy-2-hydroxypropyl)aminoethylethyleneurea, N-vinyloxyethyleneurea, N-methacryloyloxyacetoxyethylethyleneurea, N-(acrylamidoethylene)ethyleneurea, N-(methacrylamidoethylene)-ethyleneurea, 1-(2-methacryloyloxyethyl)imidazolin-2-one, and N-(methacrylamidoethyl)ethyleneurea, N-(2-methacrloyloxyethyl) ethylene urea, N-(2-methacryloxyacetamidoethyl)-N,N′-ethyleneurea, allylalkyl ethylene urea, N-methacrylamidomethyl urea, N-methacryoyl urea, N-[3-(1,3-diazacryclohexan)-2-on-propyl]methacrylamide, 2-(1-imidazolyl)ethyl methacrylate, 2-(1-imidazolidin-2-on)ethylmethacrylate, N-(methacrylamido)ethyl urea, and allyl ureido wet adhesion co-monomer.

Other functional co-monomers include, but are not limited to, acrylamide, methacrylamide, acrylonitrile, and vinyl cyanides, vinylpyrrolidone; polypropylene glycol mono(meth)acrylate or polyethylene glycol mono(meth)acrylate; silane co-monomers such as methacryloxypropyl trimethoxysilane, methacryloxypropyl triethoxysilane, methacryloxypropyl tripropoxysilane, vinyltrimethoxysilane, and vinyltriethoxysilane; crosslinkers with two or more sites of ethylenic unsaturation, such as ethylene glycol dimethacrylate, diethylene glycol dimethacrylate, trimethylolpropane trimethacrylate, 1,3-butyleneglycol dimethacrylate, and 1,4-butyleneglycol dimethacrylate.

Crosslinkable co-monomers include the following non-limiting examples: acetoacetate co-monomers containing (meth)acrylate, allyl or vinyl functional groups including but not limited to acetoacetate moieties such as: 2-acetoacetoxyethyl (meth)acrylate, 3-acetoacetoxypropyl (meth)acrylate, 4-acetoacetoxybutyl (meth)acrylate, 2-cyanoacetoxyethyl (meth)acrylate, 3-cyanoacetoxypropyl (meth)acrylate, 4-cyanoacetoxybutyl (meth)acrylate, N-(2-acetoacetoxyethyl) (meth)acrylamide, allyl acetoacetate, 2,3-di(acetoacetoxy)propyl (meth)acrylate, vinyl acetoacetate, and combinations thereof. Also suitable are co-monomers containing a keto group such as diacetone acrylamide.

Fluoropolymers and copolymers are also suitable to use as the polymer component of the waterborne coating. Non-limiting examples include polyvinylidene fluoride (PVDF) as well as fluoropolymers comprising at least 20 weight percent of one or more fluoromonomers. The term “fluoromonomer” or the expression “fluorinated monomer” means a polymerizable alkene which contains in its structure at least one fluorine atom, fluoroalkyl group, or fluoroalkoxy group whereby those groups are attached to the double bond of the alkene which undergoes polymerization. The term “fluoropolymer” means a polymer formed by the polymerization of at least one fluoromonomer, and it is inclusive of homopolymers and copolymers, and both thermoplastic and thermoset polymers. Useful fluoropolymers for use in the waterborne coating composition include, but are not limited to polyvinylidene fluoride (PVDF), ethylene tetrafluoroethylene (ETFE) polymers, terpolymers of ethylene with tetrafluoroethylene and hexafluoropropylene (EFEP), terpolymers of tetrafluoroethylene-hexafluoropropylene-vinyl fluoride (THV), polyvinylfluoride (PVF), copolymers of vinyl fluoride, and blends of PVDF with functionalized or unfunctionalized polymethyl methacrylate polymers and copolymers. The fluoropolymers may be functionalized or unfunctionalized, and could be homopolymers or copolymers—preferably copolymers with other fluorine monomers, including vinyl fluoride; vinylidene fluoride (VDF); trifluoroethylene (VF3); chlorotrifluoroethylene (CTFE); 1,2-difluoroethylene; tetrafluoro ethylene (TFE); hexafluoropropylene (HFP); perfluoro(alkyl vinyl) ethers, such as perfluoro(methyl vinyl) ether (PMVE), perfluoro(ethyl vinyl) ether (PEVE) and perfluoro(propyl vinyl) ether (PPVE); perfluoro(1,3-dioxole); perfluoro(2,2-dimethyl-1,3-dioxole) (PDD), and blends thereof.

In one preferred embodiment of the invention, the fluoropolymer is PVDF, or a copolymer of vinylidene fluoride and hexafluoropropylene.

In one embodiment of the invention, the blend of the polymer used in the emulsion or latex could be an intimate blend of two polymers, such as in an acrylic modified fluoropolymer (AMF) in which (meth)acrylate monomers are polymerized in the presence of a fluoropolymer seed.

Other Additives

Any other additives in addition to the coalescent agent described herein that are known in the art are suitable for use in waterborne latex or emulsion coating compositions comprising the low-VOC coalescent agents described herein. These can include, but are not limited to: tints, pigments, dyes or other colorants, titanium dioxide, fillers, extender pigments, dispersion aids, surfactants, foam control agents, rheology control agents, brightness enhancers, opacifiers and thickeners, freeze-thaw and/or open time additives, antioxidants or UV stabilizers. Further, it is within the scope of the invention that conventional low- and high- VOC coalescent agents can be present in the formulation in addition to the inventive coalescent agents described herein.

Test Methods

Latex viscosity

Viscosity of the latex before and after coalescent addition was measured using a Brookfield RV viscometer with a #2 spindle

Minimum Film Formation Temperature (MFFT)

The emulsion polymer was cast using a 3 mil drawdown bar (giving a 3 mil wet film thickness) over an aluminum temperature gradient bar with a temperature range of −5° C. to 15° C. The film was allowed to dry completely for at least 30 minutes as moisture was removed from the sample by a constant flow of nitrogen over the wet film. The MFFT was then measured using a thermocouple as the lowest temperature where the coating formed a clear, crack-free film. Depression of MFFT when compared to latex without coalescent is indicative of coalescent action.

Low Temperature Coalescence (LTC):

Drawdown films were prepared on Leneta B Opacity Charts using a 10 mil bird applicator. The paint films were placed in a 4.4° C. refrigerator immediately after the films were drawn down and allowed to dry for 24 hours. The dried films were visually examined for continuity. The degree of cracking on the sealed and unsealed portions of the Leneta 1B chart was rated on a 1 to 5 scale as follows:

1=severe cracking

2=moderate cracking

3=some cracking

4=slight cracking

5=no cracking

Tint Strength

Five grams of Colortrend Phthalo Blue was weighed into a half-pint can containing 250 grams of test paint. After the colorant addition, the paint can was shaken on a Red Devil shaker for 3 to 5 minutes. Paint drawdowns using the tinted paint compositions were then prepared on Leneta B charts using a 3 mil bird bar. These were allowed to dry for one day in a controlled temperature and humidity chamber at 25° C. and 50% relative humidity. The Y% brightness value was measured on a colorimeter and the percent tint strength was calculated by the Kubelka-Munk (KM) formula. In general, the higher the tint strength (the higher the Y% lightness value), the less TiO₂ is required to achieve the same hiding. The control is the sample with no coalescent.

König Pendulum Hardness

König pendulum hardness of coating films was measured following ASTM 4366. The paint films were prepared on 3 inch by 12 inch glass plates using a 10-mil drawdown bar and allowed to dry for 7 days. The dry film thickness was approximately 4 mils. The König pendulum resting on the coating surface was set into oscillation (rocking) and the time in seconds for the swing amplitude of the pendulum to decrease from 6° to 3° was recorded.

Generally, the coating that has a greater pendulum hardness is expected to exhibit higher block resistance and print resistance since pendulum hardness is related to the bulk modulus of the coating.

Scrub Resistance

Scrub resistance was measured using ASTM D2486-06, Test Method B. A laboratory control paint was used as control in the scrub test. The control and test paints were drawn down on the scrub panel, dried and then scrubbed at the same time. Scrub resistance of test paint is expressed as percentage of the scrub cycle relative to the control paint. Relative scrub resistance was evaluated on a Garner Straight Line Washability and Wear Abrasion Machine. The coatings were applied at a wet film thickness of 7 mils over Leneta black plastic charts and allowed to dry for 7 days in a controlled temperature and humidity chamber (25° C. and 50% relative humidity). The nylon bristle brushes were conditioned by running 400 cycles before the test began. A standardized abrasive scrub media (#SC-2 from the Leneta Company) was used. The test included the addition of 7 mL of scrub media and 5 mL of water at the beginning and after every 400 cycles. The experimental latex was drawn down and scrubbed side by side with an internal scrub control. The test was done in triplicate and the number of cycles to failure of the paint was recorded.

Metal Adhesion

Metal adhesion was measured according to ASTM 3359-17 Standard Test for Rating adhesion by Tape Test.

Dirt Pickup Resistance

Dirt pick up resistance was tested as follows. Aluminum panels were coated at 8 mils wet thickness and allowed to dry. Red iron oxide slurry and carbon black was applied on parts of the coating panels (typically 1 inch by 1 inch) and let sit at room condition for 4 hours. After that, the applied areas were carefully washed with water, wiped and let dry at room condition. When the washed areas were completely dried, the color difference between the exposed and unexposed areas were measured and represented by Delta E* (Hunter units). Lower Delta E* values indicate better dirt pick up resistance.

Within this specification embodiments have been described in a way which enables a clear and concise specification to be written, but it is intended and will be appreciated that embodiments may be variously combined or separated without parting from the invention. For example, it will be appreciated that all preferred features described herein are applicable to all aspects of the invention described herein.

Volatile organic content (VOC) of the coalescent agent was measured by ASTM D6886. In preferred embodiments, the VOC of the coalescent agent is no more than 0.5%, preferably less than 0.2%, more preferably less than 0.1%, even more preferably less than 0.01%, by weight of the coalescent agent.

### EXAMPLES

Several inventive coalescing agents were synthesized and are summarized in Table 1 below. Coalescent Agent A is a Comparative. Coalescents B through J are of the invention.

Optifilm 400 (Comparative Coalescent X), Benzoflex® LC-531 (Comparative Coalescent Y) (both from Eastman Chemical), and Loxanol CA 5310 (Comparative Coalescent Z) (BASF), three commercially available low-VOC coalescent and plasticizers used in low-VOC waterborne coatings, caulks and sealants formulations, were included as benchmarks.

TABLE 1
Composition of Coalescent Agents

| Coalescing Agent | Source Oil | Ester group | Functional group |
|---|---|---|---|
| Coalescent A | Soybean | Methyl | Epoxy |
| (Comparative) | | | |
| Coalescent B | Soybean | Butyl | Epoxy |
| Coalescent C | Soybean | Ethylhexyl | Epoxy |
| Coalescent D | Canola | Methyl | Epoxy |
| Coalescent E | Canola | Butyl | Epoxy |
| Coalescent F | Linseed | Methyl | Epoxy |
| Coalescent G | Soybean | Methyl | Vicinal diol |
| Coalescent H | Soybean | Butyl | Vicinal diol |
| Coalescent I | Canola | Methyl | Vicinal diol |
| Coalescent J | Soybean | Methyl | Hydroxy ester |

### Example 1: Coalescent Efficiency in Latex

The efficiency of the coalescent agents is assessed by depression of minimum film forming temperature (MFFT) and Tg of polymers containing various inventive and comparative coalescent agents. A pre-determined amount of coalescent was added to Encor® 662 (50% solids, all-acrylic polymer latex from Arkema) or Encor® 379G (55% solids, vinyl acetate-butyl acrylate copolymer latex from Arkema) or a representative styrene acrylic latex under mixing. These materials were selected to represent the major classes of emulsion polymers used in architectural paints.

The results shown below in Tables 2 to 4 indicate that the epoxidized compositions of the present invention are comparable coalescents to the benchmark coalescent agents. The results shown in Tables 2 to 4 are for the latex compositions with no other coating ingredients.

TABLE 2
Minimum Film Formation Temperature (MFFT) of
Encor ® 662 with coalescent at 8 wt % on dry polymer solids

| Coalescent | MFFT (° C.) |
|---|---|
| none | 15 |
| Comparative Coalescent X | −1.8 |
| Comparative Coalescent Z | −0.1 |
| Coalescent A | −0.9 |
| Coalescent B | −0.5 |
| Coalescent D | 0.8 |
| Coalescent E | 0.4 |
| Coalescent F | 0.6 |
| Coalescent G | 2.5 |

TABLE 3
Minimum Film Formation Temperature (MFFT) of
Encor ® 379G with coalescent at 8 wt % on dry polymer solids.

| Coalescent | MFFT |
|---|---|
| none | 8.6 |
| Comparative Coalescent X | −0.2 |
| Comparative Coalescent Z | −0.1 |
| Coalescent A | −2.9 |
| Coalescent B | 1.4 |
| Coalescent C | 3 |
| Coalescent D | −2.3 |
| Coalescent E | 1.1 |
| Coalescent F | −1.2 |
| Coalescent G | −0.2 |
| Coalescent H | 3.3 |
| Coalescent I | 1.7 |
| Coalescent J | 3.7 |

TABLE 4
Minimum Film Formation Temperature (MFFT) of styrene
acrylic latex with coalescent at 8 wt % on dry polymer solids

| Coalescent | MFFT (° C.) |
|---|---|
| neat latex | >13 |
| Comparative Coalescent X | −1.6 |
| Comparative Coalescent Z | −0.1 |
| Coalescent A | −0.5 |
| Coalescent B | 1.7 |
| Coalescent D | −1.2 |
| Coalescent E | 0.4 |
| Coalescent F | 1.4 |

All inventive and comparative coalescents imparted a substantial depression of the MFFT compare to the latex without coalescent agent present. This MFFT depression is indicative of efficacy as a coalescing agent in the given latex.

### Example 2: Paint Formulations Using Inventive Coalescent Agent

The epoxidized coalescent agents were compared to the commercial low-VOC benchmark in a flat paint formulation using Encor® 636 as the polymeric binder. Tables 5 and 6 together show the complete flat paint formulations. The same grind formulation, which is shown in Table 5 has added to it the additional ingredients as listed in Table 6

TABLE 5
Flat paint formulation, except for
latex and Coalescent Agent F (of the invention)

| Grind-2 | Weight (lbs) |
|---|---|
| Water | 280.00 |
| Natrosol 250 HBR | 2.00 |
| Ecodis P50 (40%) | 10.00 |
| Foamstar ST 2436 | 1.00 |
| TI-PURE 706 | 220.00 |
| Minex 4 | 215.00 |
| AMP-95 | 1.00 |
| Proxel GXL | 3.01 |
| Grind Sub Total | 732.01 |

TABLE 6
Flat Individual Thindowns (Coalescent Agent F (of the invention))

| Weights (lbs) | | | | |
|---|---|---|---|---|
| Paint Samples | P1 | P2 | P3 | P4 |
| Common Grind | 732.01 | 732.01 | 732.01 | 732.01 |
| ENCOR 636 (50%) | 300.00 | 300.00 | 300.00 | 300.00 |
| Comparative Coalescent | | 7.46 | | |
| X (wt % by polymer | | (10.5%) | | |
| content) | | | | |
| Comparative Coalescent Z | | | 7.46 | |
| (wt % by polymer content) | | | (10.5%) | |
| Coalsecent F | | | | 7.46 |
| (wt % by polymer content) | | | | (10.5%) |
| Water | 95.00 | 95.00 | 95.00 | 95.00 |
| Rheotech 3800 | 6.00 | 6.00 | 6.00 | 6.00 |
| Foamstar ST 2436 | 1.00 | 1.00 | 1.00 | 1.00 |
| Coapur 2020 | 21.00 | 21.00 | 21.00 | 21.00 |
| Total | 1155.01 | 1162.47 | 1162.47 | 1162.47 |

Low temperature coalescence (LTC) measures the goodness or completeness of coalescence of latex paints at a low temperature. The LTC results in Table 7 confirms that the epoxidized fatty acid esters when used as coalescent agents in accordance with the present invention have equal or better coalescing efficiency to the leading commercial low-VOC coalescent.

TABLE 7
LTC of flat acrylic paint based on Encor ® 636

| Paint # | P1 | P2 | P3 | P4 |
|---|---|---|---|---|
| Coalescent | — | 10.5% | 10.5% | 10.5% |
| Agent | | Comparative | Comparative | Coalescent F |
| | | Coalescent X | Coalescent Z | (of the invention) |
| LTC: Sealed | 2 | 4.5 | 3.5 | 5 |
| LTC: Unsealed | 2 | 5 | 3.5 | 5 |

### Example 3: Scrub Resistance and Tint Strength

Other paint performance properties are compiled in Table 8. The low VOC coalescent agents of the present invention provide equivalent or improved scrub resistance and tint strength compared to the results obtained using the commercial benchmark low VOC coalescent agents.

TABLE 8
Scrub Resistance and Tint Strength
of Acrylic Paint Based on Encor ® 636

| Paint # | P2 | P3 | P4 |
|---|---|---|---|
| Coalescent | 10.5% | 10.5% | 10.5 |
| | Comparative | Comparative | Coalescent F |
| | Coalescent | Coalescent | (of the |
| | X | Z | invention) |
| Scrub Resistance, | 91% | 97% | 96% |
| % control | | | |
| KM Tint Strength, | 101.4% | 101.7% | 101.41% |
| % control | | | |

### Example 4: Heat Stability in Styrene Acrylic Latexes

The following examples demonstrate broad compatibility of the inventive coalescent with various binder chemistries commonly utilized in waterborne coatings including styrene acrylic latexes. A large increase of latex viscosity over time is an indication of poor storage stability.

TABLE 9
Storage stability of Encor ® 423 styrene acrylic

| | Coalescent | | | | |
|---|---|---|---|---|---|
| | Compar- | | Compar- | Compar- | |
| | ative | Glycol | ative | ative | Coalescent |
| | Coalescent | Ether | Coalescent | Coalescent | C (of the |
| | X | DPM | Y | Z | invention) |
| Initial | 956 | 128 | 371 | 723 | 739 |
| Viscosity | | | | | |
| (cPs) | | | | | |
| Viscosity | 3000 | 128 | 366 | 807 | 1172 |
| (cPs) after | | | | | |
| 3 weeks | | | | | |
| Vicosity | 2044 | 0 | −5 | 84 | 433 |
| change, | | | | | |
| cPs | | | | | |

The paint formulation used to evaluate the epoxidized linseed oil-based coalescent C along with two commercial coalescents is given in Table 10. Coalescent C provided similar is coalescent efficiency as all paints passed LTC at from 0 to 60 mil thickness. Table 11 shows that good paint stability was maintained as indicated by constant gloss and KU viscosity after heat aging at 60 C for 4 weeks. Moreover, Coalescent C yielded the highest Konig harness values.

TABLE 10
Paint formulation based on Encor ® 423

| | 1x | | |
|---|---|---|---|
| | Weight | 1x Vol | |
| Common Grind | | | |
| Tap water | 70.0 | 8.5 | |
| Byk 022 | 2.0 | 0.1 | |
| AMP-95 | 2.0 | 0.2 | |
| Coadis 123K | 8.0 | 0.9 | |
| Ecodis P50 | 3.1 | 0.3 | |
| Surfynol 104DPM | 5.0 | 0.3 | |
| Titanium dioxide R-706 | 150.0 | 4.5 | |
| Nicron 503 | 200.0 | 8.9 | |
| Coapur ® 2020 | 8.0 | 0.9 | |
| Common Thindown | | | |
| Byk 024 | 2.0 | 0.2 | |
| TSP-16 Surfactant | 2.0 | 0.2 | |
| Acticide ® MBS | 2.5 | 0.3 | |
| Coapur ® 817W | 5.0 | 0.6 | |
| Totals of Common Paste: | 459.6 | 26.0 | |
| Individual Thindowns | | | |
| Grind | 459.6 | 459.6 | 459.6 |
| Encor 423 | 500.0 | 500.0 | 500.0 |
| Glycol Ether DPM | 26.0 | 26.0 | 26.0 |
| Comparative Coalescent Y | 30.0 | | |
| Comparative Coalescent Z | | 30.0 | |
| Vikoflex 9010 | | | 30.0 |
| Water | 177.2 | 177.2 | 177.2 |
| Tot. Weight | 1192.8 | 1192.8 | 1192.8 |
| Tot Volume | 112.0 | 112.0 | 112.0 |

TABLE 11
Heat age stability, adhesion and Konig hardness of Encor ® 423 paints

| | | Comparative | Comparative | Coalescent |
|---|---|---|---|---|
| | | Coalescent | Coalescent | C (of the |
| | | Y | Z | invention) |
| Overnight | KU | 74 | 78 | 79 |
| equilibration | 60-degree gloss | 11.4 | 11.4 | 11.9 |
| After 4- | KU | 69 | 72 | 77 |
| weeks at 60 C. | 60-degree gloss | 11.1 | 12.4 | 12.5 |
| metal | Steel | 5B | 5B | 5B |
| adhesion | Treated | 5B | 5B | 5B |
| | Aluminum | | | |
| | Untreated | 5B | 5B | 5B |
| | aluminum | | | |
| | Galvanized steel | 5B | 5B | 5B |
| Konig | 1 day | 40 | 28 | 60 |
| Hardness | 3 day | 58 | 31 | 67 |
| | 7 day | 64 | 29 | 71 |

### Example 5: Coalescent Performance in Fluorpolymer-acrylic Latexes

Fluoropolymer acrylic based paints were made from formulas shown in Tables 12 and 13.

TABLE 12
Masterbatch Pigment Grind

| | Grams |
|---|---|
| water | 180.51 |
| Byk 190 | 17.57 |
| Ammonia 28% | 0.08 |
| Byk 022 | 1.25 |
| PK 0 VOC | 11.74 |
| KTPP | 0.8 |
| Natrasol 250 MBR | 4.1 |
| Minex 7 | 164.79 |
| TiO2 R 960 | 172.49 |

TABLE 13
Individual thindowns

| | P5 | P6 | P7 | P8 |
|---|---|---|---|---|
| Pigment grind | 369 | 369 | 369 | 369 |
| Kynar ® FMA 12 | 384.9 | 384.9 | 384.9 | 384.9 |
| BYK 022 | 0.61 | 0.61 | 0.61 | 0.61 |
| DPnB | 13.1 | 13.1 | 13.1 | |
| Comparative Coalescent X | 27.45 | | | |
| Coalescent D (of the invention) | | 27.45 | | |
| Coalescent F (of the invention) | | | 27.45 | |
| Coalescent A (comparative) | | | | 27.45 |
| Coapur XS 71 | 2.73 | 2.73 | 2.73 | 2.73 |
| Coapur XS 22 | 1.45 | 1.45 | 1.45 | 1.45 |

Paints were made from these formulas and placed at room temperature. KU viscosity readings were taken at initial, next day, and 2 week intervals. Table 14 below summarizes room temperature paint stability and MFFT of these paints. Results of dirt pick up testing are shown in table 15.

TABLE 14
Paint stability and MFFT

| KU | | | | |
|---|---|---|---|---|
| | Initial | 24 hrs | 14 day | MFFT |
| P5 | 108.2 | 107.5 | 109.2 | <0 |
| P6 | 105.9 | 105.8 | 108.9 | <0 |
| P7 | 105.4 | 105.1 | 109.5 | <0 |
| P8 | 107.1 | 106.6 | 110 | <0 |

TABLE 15
Dirt pick up test data
Dirt Pick up ΔE* value

| | red | black |
|---|---|---|
| Competitive Coalescent X | 1.1 | 5.3 |
| Coalescent D | 3.2 | 0.65 |
| Coalescent F | 1.1 | 2.4 |
| Coalescent A (comparative) | 2.9 | 1.8 |

In some embodiments, the invention herein can be construed as excluding any element or process that does not materially affect the basic and novel characteristics of the composition or process. Additionally, in some embodiments, the invention can be construed as excluding any element or process not specified herein.

Although the invention is illustrated and described herein with reference to specific embodiments, the invention is not intended to be limited to the details shown. Rather, various modifications may be made in the details within the scope and range of equivalents of the claims and without departing from the invention.

1. A coalescent agent for waterborne coatings, wherein the coalescent agent comprises one or more functionalized fatty acid C1-C22 alkyl or benzyl esters, preferably C2-C8 alkyl or benzyl esters, wherein said functionalized fatty acid esters have one or more functional groups per molecule selected from the group consisting of epoxides, excluding coalescent agents of epoxidized fatty acid methyl esters obtained from soybean oil where the alkyl is Cl (methyl epoxy esters derived from soybean oil).

2. The coalescent agent of claim 1, wherein said functionalized fatty acid esters are selected from the group consisting of monoesters, fatty acid monoglycerides, fatty acid esters of aliphatic mono-alcohols, fatty acid esters of aromatic alcohols, fatty acid esters of benzyl alcohol, fatty acid diesters, fatty acid diglycerides, fatty acid esters of diols wherein both hydroxyl groups are esterified with fatty acid, fatty acid triesters, fatty acid triglycerides, fatty acid esters of triols other than glycerin in which all three hydroxyl groups are esterified with fatty acid, fatty acid esters of polyols containing more than three hydroxyl groups per molecule, or mixtures thereof and preferably is a monoester, monoglyceride, diglyceride, or mixtures thereof.

3. The coalescent agent of claim 1, wherein said functionalized fatty acid esters are derived from one or more vegetable or animal oils selected from the group consisting of algae oil, avocado oil, canola oil, coconut oil, castor oil, corn oil, cottonseed oil, flax oil, fish oil, grapeseed oil, hemp oil, jatropha oil, jojoba oil, mustard oil, dehydrated castor oil, palm oil, palm stearin, rapeseed oil, safflower oil, soybean oil, sunflower oil, tall oil, olive oil, tallow, lard, chicken fat, tung oil, linseed oil, linoleic oil, peanut oil, coconut oil, and mixtures thereof.

4. The coalescent agent of claim 1, wherein said functionalized fatty acid esters have an oxirane value of 1 to 10 weight percent of oxirane oxygen, an iodine number of no more than 4 g 12/100 g and an acid value of no more than 10 mg KOH/g.

5. The coalescent agent of claim 1 wherein the oxirane value of said epoxidized fatty acid esters is between 4 and 10 weight percent oxirane oxygen.

6. The coalescent agent of claim 1 wherein the volatile organic content (VOC) of the coalescent agent as measured by ASTM D6886 is no more than 0.5% by weight of the coalescent agent.

7. The coalescent agent of claim 1 wherein said one or more functionalized fatty acid alkyl or benzyl esters are epoxidized fatty acid alkyl or benzyl esters.

8. The coalescent agent of claim 1, wherein the coalescent agent comprises epoxidized fatty acid C1-C22 alkyl esters.

9. The coalescent agent of claim 1, wherein the coalescent agent comprises epoxidized fatty acid C1-C8 alkyl esters.

10. The coalescent agent of claim 1 wherein said coalescent agent is derived from soybean oil, canola oil or blends thereof.

11. The coalescent agent of claim 1 wherein said functionalized fatty acid esters are epoxidized fatty acid monoglycerides or functionalized fatty acid diglycerides.

12. The coalescent of claim 1 where the coalescing agent is derived from soybean oil with C2 to C8 alkyl esters.

13. The coalescent agent of claim 1 wherein said functionalized fatty acid esters are epoxidized fatty acid monoglycerides or epoxidized fatty acid diglycerides.

14. A waterborne coating composition comprising the coalescent agent according to claim 1 and a polymeric resin emulsion.

15. The waterborne coating composition of claim 14 wherein said polymeric resin emulsion is comprised of at least one polymeric resin selected from the group consisting of vinyl acetate homopolymers, vinyl acetate copolymers, acrylic homopolymers, acrylic copolymers, vinyl acetate ethylene copolymers, fluoropolymers, acrylic modified fluoropolymers, blends of fluoropolymers with acrylic polymers, and styrene acrylic copolymers.

16. The waterborne coating composition of claim 14 wherein said coalescent agent is present in the composition at a level of between 1% and 20% by weight of the dry polymeric resin in the composition.

17. The waterborne coating composition of claim 14 wherein said coalescent agent is present in the composition at a level of between 2% and 12% by weight of the dry polymeric resin in the composition.

18. A method of improving a coalescent efficiency of a waterborne polymeric emulsion wherein the method comprises blending at least 1% to 20%, by weight of the dry polymeric resin in the emulsion, of the coalescent agent according to claim 1 into the polymeric resin emulsion, and wherein the coalescent efficiency is determined by a reduction in the glass transition temperature of the polymeric emulsion and by a reduction in the minimum film-forming temperature of the polymeric resin emulsion.

19. The method of claim 18, wherein the reduction in the glass transition temperature is at least 1° C. and the reduction in the minimum film-forming temperature is at least 1° C.

20. The method of claim 18, wherein the reduction in the glass transition temperature is at least 4° C. and the reduction in the minimum film-forming temperature is at least 4° C.

21. (canceled)

22. (canceled)
